(19) United States
(12) Patent Application Publication
(10) Pub. No.: US 2014/0163111 A1
(43) Pub. Date: Jun. 12, 2014

(54) POROUS BIOMOLECULE-CONTAINING METAL-ORGANIC FRAMEWORKS

(71) Applicant: UNIVERSITY OF PITTSBURGH - OF THE COMMONWEALTH SYSTEM OF HIGHER EDUCATION, PITTSBURGH, PA (US)

(72) Inventors: NATHANIEL LOUIS ROSI, PITTSBURGH, PA (US); JIHYUN AN, EVANSTON, IL (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 14/179,922

(22) Filed: Feb. 13, 2014

Related U.S. Application Data
(63) Continuation-in-part of application No. 12/915,178, filed on Oct. 29, 2010.
(60) Provisional application No. 61/256,487, filed on Oct. 30, 2009.

(51) Int. Cl. B01J 39/04; A61K 31/166; C07F 3/06

(52) U.S. Cl. CPC B01J 39/04; C07F 3/06; A61K 31/166
USPC 514/619; 544/226; 423/437.1; 423/648.1; 423/351

(57) ABSTRACT

The invention relates to compositions including porous biomolecule-containing metal-organic frameworks and methods for their preparation. The porous biomolecule-containing metal-organic frameworks can include a metal component and a biomolecule component. The pores located within the frameworks have a pore space and said pore space is capable to adsorb materials therein. These compositions of the invention are useful in a wide variety of applications, such as, but not limited to, hydrogen and carbon dioxide sequestration, separation and storage; carbon dioxide uptake; and drug storage and release.

### CROSS-REFERENCE TO RELATED APPLICATION

This application is a continuation-in-part application and claims a benefit of priority from U.S. patent application Ser. No. 12/915,178, filed Oct. 29, 2010, and entitled “Porous Biomolecule-Containing Metal-Organic Frameworks,” which claims a benefit of priority from U.S. Provisional Patent Application Ser. No. 61/256,487, filed Oct. 30, 2009, and entitled “Porous Biomolecule-Containing Metal-Organic Frameworks”, which are herein incorporated by reference in their entirety.

### BACKGROUND OF THE INVENTION

1. Field of the Invention

The invention relates to compositions including porous biomolecule-containing metal-organic frameworks; porous, biomolecule-containing metal-organic macrocyclic structures; and the like; and methods for their preparation. These compositions are useful in a wide variety of applications, in particular, hydrogen and carbon dioxide capture, separation and storage; carbon dioxide uptake; and drug storage and release.

2. Background Information

The metal-organic frameworks (“MOFs”) known in the art are crystalline compounds which consist of inorganic secondary building units (e.g. metal clusters or metal ions) linked together with organic molecules to form one-, two-, or three-dimensional structures. MOFs are also known as hybrid frameworks and coordination polymers. MOFs are used in applications such as gas purification, gas separation, catalysis and sensors. MOF-5 is a known matrix which is built up by Zn₄O groups on the corners of a cubic lattice, connected by 1,4-benzenedicarboxylic acid. MOF-74 is a known matrix composed mainly of carbon and zinc.

Frameworks can be prepared by reflux, precipitation, and recrystallization processes. For example, reagents can be sealed in an autoclave with water or solvent and heated to a temperature of from 100° C. to 250° C. This allows for the activation energy needed to assemble the complex framework structures to be achieved without the solvent evaporating.

It would be desirous for the metal-organic frameworks to have pores and/or channels structured therein such that gas and/or other material may be adsorbed or trapped within the spaces. Further, it would be advantageous for the spaces within the pores to be adjustable and controllable. These porous materials constructed from metal ions and organic molecular building blocks can be used for a variety of applications. For example, for clean energy applications, porous metal-organic structures can be used to adsorb and trap large amounts of carbon dioxide (CO₂) and hydrogen (H₂) gases; for drug delivery applications, porous metal-organic structures can be used to store and release drug molecules such that the drug can be administered in a controlled manner over a period of time. The properties of such materials can be adjusted based on the selection of the metal ions and the molecular building blocks.

### BRIEF DESCRIPTION OF THE DRAWINGS

A further understanding of the invention can be gained from the following description of the preferred embodiments when read in conjunction with the accompanying drawings in which:

FIG. 1 is a diagram showing the chemical structure of a zinc-adeninate macrocycle, in accordance with certain embodiments of the invention;

FIG. 2 is a diagram showing zinc-adeninate building units connected by biphenyldicarboxylate ligands which exhibit void spaces formed therein, in accordance with certain embodiments of the invention;

FIG. 3 is diagram showing the three-dimensional framework of a cobalt-adeninate metal-organic framework which exhibits pores that are densely lined with pyrimidal nitrogens and amino groups from the adenine building blocks, in accordance with certain embodiments of the invention;

FIG. 4 is a diagram showing chemical structures of suitable carboxylic acids for use in preparing neutral frameworks, in accordance with certain embodiments of the invention;

FIG. 5 is a plot showing carbon dioxide adsorption isotherms of acetate, propionate, butyrate and valerate analogues, in accordance with certain embodiments of the invention;

FIG. 6 is a plot showing carbon dioxide adsorption isotherms of acetate, cyclopropylacetate, isovalerate and valerate analogues, in accordance with certain embodiments of the invention; and

FIG. 7 is a plot of powder x-ray diffraction data showing water stability of acetate, propionate, butyrate, and valerate analogues, in accordance with certain embodiments of the invention.

### SUMMARY OF THE INVENTION

In an aspect, the invention provides a composition including a biomolecule-containing metal-organic framework having pores structured therein, said framework including a metal component and a biomolecule component, said pores structured to have a pore volume and to adsorb material therein, and said pore volume adjusted by a mechanism selected from the group consisting of cation exchange of cations in the pores with different cations and pre-selection of a pendant group R on an aliphatic chain of a framework comprising monocarboxylate having a structure (M)₂(BC)₂(OOC—R)₂ wherein M is the metal component, BC is the biomolecule component and R is alkyl. The metal component can be selected from the group consisting of metal cluster, metal ion or combination thereof. Further, the metal component can be selected from the group consisting of Cu, Ti, Cr, Fe, Ni, Mn, Co, Zn, Zr, Al, In, and salts and mixtures thereof, and R can be C₁ to C₈ alkyl. The biomolecule can include a nucleobase. The nucleobase can be selected from the group consisting of adenine, guanine, cytosine, thymine, uracil and mixtures thereof. The biomolecule can be biologically compatible with the metal component.

The aforementioned composition can further include an organic component. The organic component can include an organic ligand. The organic ligand can include a multitopic material. The organic ligand can be selected from the group consisting of dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures and combinations thereof.

The framework can be selected from the group of structures consisting of macrocyclic structures, crystalline structures, polyhedra, extended framework structures, and combinations thereof. The framework can include a plurality of units connected by a linker. The linker can be selected from 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid; 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; 4,4′,4,4″-[benzene-1,3,5-triyl-tris(benzene-4,1-diyl)]tribenzoate; azobenzene-4,4′-benzenedicarboxylate; and mixtures thereof.

In one embodiment, the metal component includes zinc salt or a cobalt salt. In another embodiment, the nucleobase includes adenine.

The pores can adsorb material selected from the group consisting of gas, drug, protein, polymer and combinations thereof. The gas can be selected from the group consisting of carbon dioxide, hydrogen, nitrogen, and mixtures thereof. The material can be a drug and the framework can controllably release the drug from the pores.

The biomolecule-containing metal-organic framework can have a BET surface area from about 1,000 to about 4,500 m²/g. Further, the biomolecule-containing metal-organic framework can have a pore volume from about 2 to about 6 cm³/g or about 4.3 cm³/g.

In another aspect, the invention provides a method for preparing a biomolecule-containing metal-organic framework comprising reacting a metal component and a biomolecule component, the framework having pores structured therein, the pores structured to have a pore volume and to adsorb materials therein, and said pore volume adjusted by a mechanism selected from the group consisting of cation exchange of cations in the pores with different cations and pre-selection of a pendant group R on an aliphatic chain of a framework comprising monocarboxylate having a structure (M)₂(BC)₂(OOC—R)₂ wherein M is the metal component, BC is the biomolecule component and R is alkyl. The metal component can be selected from the group consisting of Cu, Ti, Cr, Fe, Ni, Mn, Co, Zn, Zr, Al, In, and salts and mixtures thereof R may be C₁ to C₅ alkyl.

In another aspect, the invention provides a method for storing and controllably releasing drug material including the aforementioned composition.

In still another aspect, the invention provides a method for capturing and storing carbon dioxide material including the aforementioned composition.

### DETAILED DESCRIPTION OF THE INVENTION

The compositions of the invention include biomolecule-containing porous metal-organic frameworks (MOFs) composed of metal and biomolecule building units. As used herein and the claims, the term “porous metal-organic framework” includes biomolecule-containing porous metal-organic macrocycles, macrocyclic structures, crystalline structures, polyhedra and extended framework structures. The MOFs of the invention include anionic and neutral frameworks. The porous metal-organic frameworks include a metal component and a biomolecule component. The metal component includes metal clusters, metal ions, or combinations thereof. The metal clusters and/or metal ions can serve as vertices. Non-limiting examples of suitable metal components include Cu, Ti, Cr, Fe, Ni, Mn, Co, Zn, Zr, Al, In, and salts and mixtures thereof. In certain embodiments, the metal component includes zinc (Zn) ions. The biomolecule component includes a nucleobase. Non-limiting examples of suitable nucleobases include, but are not limited to, adenine, guanine, cytosine, thymine, uracil and mixtures thereof. In certain embodiments, the nucleobase is adenine. The metal component and biomolecule component can be biologically compatible such that they can be used as building blocks for the construction of discrete metal-biomolecule macrocycles, macrocyclic structures, crystalline structures, polyhedra, and extended framework structures. In certain embodiments, the framework can include more than one macrocycle. In further embodiments, the macrocytes can be connected by hydrogen bonds.

The porous metal-organic frameworks (MOFs) of the invention can also include an organic component. The organic component includes organic ligand. The organic ligand can serve as a linker. The linker(s) can connect vertices into structures. The functionality of the framework can be tuned by selecting particular metal and organic components. The metrics of the framework can be tuned by modulating the length of the organic linker molecule. Organic ligands, such as organic carboxylic acids, and salts or esters thereof, e.g., monocarboxylates, can be used for MOFs. The organic ligand may be multitopic, such as, but not limited to, ditopic, tritopic, and tetratopic. As used herein, the term “topicity” refers to the number of functional groups present that can bind a metal. For example, a ditopic ligand has two functional groups capable of binding a metal, a tritopic ligand has three functional groups and a tetratopic ligand has four functional groups. Non-limiting examples of suitable organic ligands for use in the invention include monocarboxylate ligands, dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures or combinations thereof.

In certain embodiments, the metal component is zinc salt or a cobalt salt. In other embodiments, the nucleobase is adenine.

The invention also provides a method for preparing a porous metal-organic framework. The method includes reacting the metal component, the biomolecule component, and optionally the organic component.

In certain embodiments, adenine is generally reacted with metal salt under solvothermal conditions using various reactant concentrations, solvents, pH, pressure, temperatures and reaction times. Non-limiting examples of suitable metal salts include nickel and palladium for synthesizing MOFs. Further, other metal salts have been combined with ugands, such as divalent zinc and cobalt to synthesize zeolite structures.

In certain embodiments, the reaction of metal cation and adenine is carried out such that coordination occurs between the metal cation and the imidazole ring of adenine.

The biomolecule-containing porous MOFs of the invention can be of various shapes and sizes. The frameworks can be one-, two-, or three-dimensional structures. The frameworks can be neutral or anionic. Moreover, the porous MOFs exhibit pores and/or channels throughout the structure. The inside of the pores and/or channels can be neutral or cationic. A variety of cations can be present within the pores, such as, but not limited to, dimethylammonium. The size, shape, volume and number of pores and/or channels can vary. The space or volume within the pores and/or channels is useful to adsorb and store material. The material can include gas (e.g., molecules), such as, but not limited to, hydrogen, carbon dioxide, nitrogen, and mixtures thereof; drug (e.g., molecules); proteins; polymers; and mixtures or combinations thereof. The stored material in the pore space or volume can then be released in a controlled manner from the frameworks.

The size, volume and functionality of the pores can be controlled by various mechanisms or processes. In certain embodiments, pore size and volume are controlled by a synthesis mechanism. In this mechanism, a monocarboxylate is present and the aliphatic carbon chain and pendant group thereon are varied or modified, e.g., pre-selected, in order to adjust or tune the pore size and volume as well as the functionality of the resulting framework. For example, varying the aliphatic carbon chain can allow systematic tuning, e.g., optimization, of CO₂ adsorption behavior. In certain embodiments, wherein the pore volume is increased, the pore volume can be about 4.3 cm³/g or higher. Furthermore, the BET surface area can be modified (e.g., tuned) within the pores. For example, the BET surface area may increase or decrease. In alternate embodiments, wherein the BET surface area is increased, the BET surface area can be about 4500 m²/g or higher.

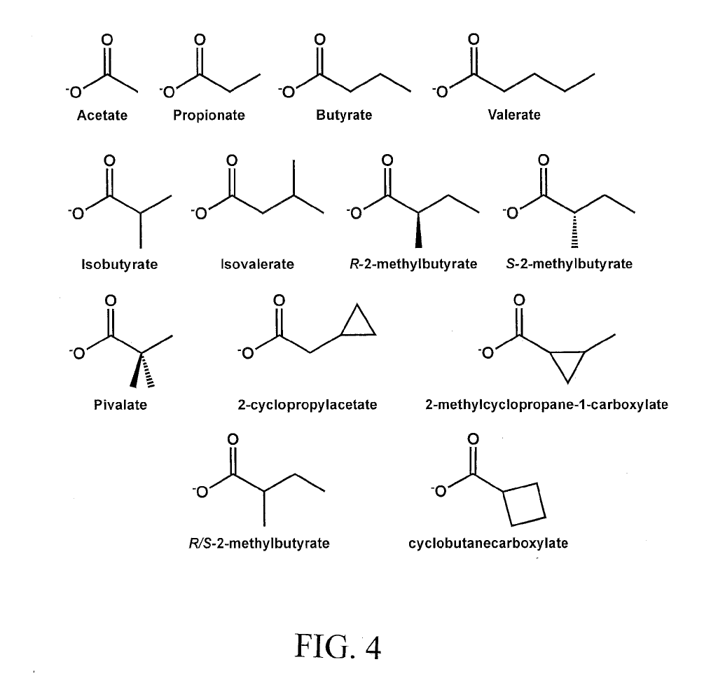

In certain embodiments, the framework of the invention including a monocarboxylate has the structure: M₂(adeninate)₂(OOC—R)₂ wherein M is a metal such as Cu, Ti, Cr, Fe, Ni, Mn, Co, Zn, Zr, Al, In, and salts and mixtures thereof. R is branched or un-branched alkyl. In certain embodiments, R can be branched or un-branched C₁₀₅ alkyl. Further, R can include a functional group such as a hydroxyl group (OH). The oxygen substituent is pendent to the first carbon (C₁) in the carbon chain. As shown in FIG. 4, non-limiting examples of suitable (OOC—R) groups include acetate, propionate, butyrate, valerate, isobutyrate, isovalerate, R-2-methylbutyrate, S-2-methylbutyrate, pivalate, 2-cyclopropylacetate, 2-methylcyclopropane-1-carboxylate, R/S-2-methylbutyrate, cyclobutanecarboxylate, and mixtures thereof.

In other embodiments, pore size and volume are controlled by cation exchange. Further, the functionality of the pores, such as, the capability to adsorb gas or other materials, also can be controlled by synthesis, and cation exchange. As used herein, the term “cation exchange” refers to replacing cations located in the pores with different cations. For example, the cation exchange can include the exchange or replacement of cations located inside the pores, such as, but not limited to, dimethylammonium cations with different cations, such as, but not limited to, tetramethylammonium cations, tetramethylammonium cations, tetraethylammonium cations, tetrabutylammonium cations, and mixtures thereof. The tetramethylammonium cations, tetraethylammonium cations and tetrabutylammonium cations are slightly larger in size than the dimethylammonium cations.

In certain embodiments, at least a portion of the cations in the pores are exchanged or replaced with larger cations. The larger cations will occupy a greater amount of space within the pores and therefore, the remaining (e.g., open) pore volume can be reduced. In alternate embodiments, the cations located within the pores are exchanged with cations having a smaller size. The smaller cations will occupy a lesser amount of space within the pores and therefore, the remaining (e.g., open) pore volume can be increased. The exchange of cations located inside the pores with different cations (e.g., larger or smaller) allows for the pore volume to be modified (e.g., tuned) in accordance therewith.

In certain embodiments, wherein the metal component is zinc salt or a cobalt salt, the biomolecule component is adenine, and the organic component is a dicarboxylate ligand, the framework can include zinc-adeninate octahedral units connected with a biphenyldicarboxylate linker to form a three-dimensional structure that can be either microporous or mesoporous. The framework can include cobalt-adeninate building units and can have Lewis basic sites (amino and pyrimidil) from the adenine exposed to the pores. In alternate embodiments, the linker can be selected from a variety of materials known in the art. The linkers can include linear linkers, trigonal linkers and mixtures thereof. Non-limiting examples of linear linkers include, but are not limited to, 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid, and mixtures thereof. Non-limiting examples of trigonal linkers include, but are not limited to, 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; 4,4′,4,4″-[benzene-1,3,5-triyl-tris(benzene-4,1-diyl)]tribenzoate; azobenzene-4,4′-benzenedicarboxylate; and mixtures thereof. Without intending to be bound by any particular theory, it is believed that increasing the length and/or size of the linker used to connect units within the framework, can result in an increase in pore volume. For example, the use of terphenyl dicarboxylate can produce a mesoporous framework having a higher pore volume than the pore volume of a mesoporous framework using biphenyl dicarboxylate linker. In alternate embodiments, a mesoporous framework in accordance with the invention can include a pore volume of from about 2 to about 4.3 cm³/g or from about 4.3 to about 6 cm³/g or from about 2 to about 6 cm³/g. In one embodiment, the pore volume is about 4.3 cm³/g.

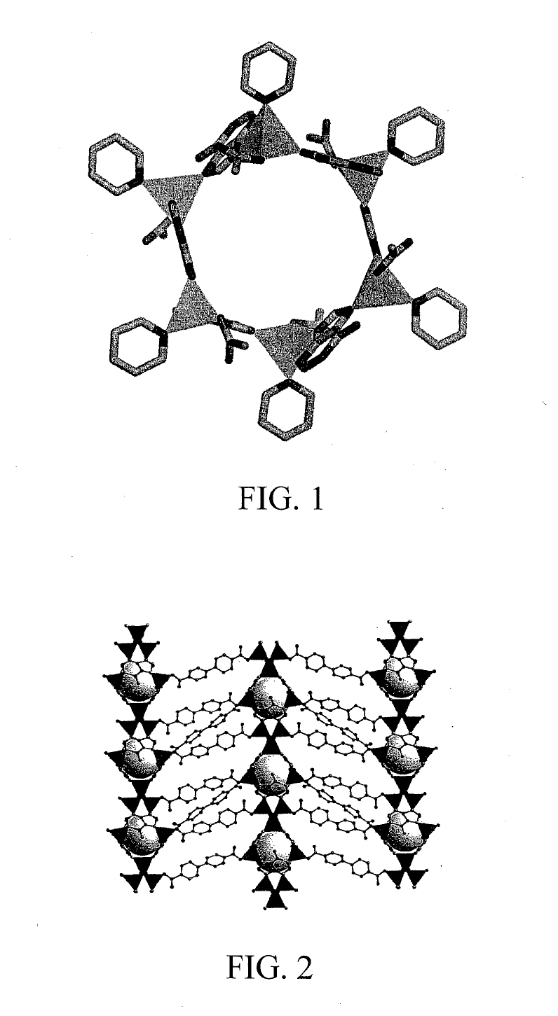

In certain embodiments, a porous metal-organic framework in accordance with the invention may be formed using single crystal growth methods. For example, the metal compound, such as zinc salt, is reacted with the nucleobase, such as adenine, at room temperature in the presence of solvent (such as, dimethylformamide (DMF), pyridine and mixtures thereof), under solvothermal conditions (such as, heating) to produce discrete zinc-adeninate macrocycles. Adeninate can coordinate metal ions through any of its five nitrogens. The macrocycles can be hexameric (e.g., containing six subunits or moieties), as shown in FIG. 1. The hexameric macrocyle, in FIG. 1, contains six zinc tetrahedra, each at a corner of the hexagon, and six adeninate ligands, at the edges of the hexagon which bridge the zinc tetrahedra together through imidazole nitrogens. The remaining two coordination sites on the zinc are occupied by pyridine and acetate. For example, six Zn²⁺ occupy the vertices of the macrocycle and adeninates bridge the Zn²⁺ through their imidazolate nitrogens. Each Zn²⁺ binds in a tetrahedral fashion to two adeninates, one pyridine molecule, and one dimethylcarbamate anion (formed in-situ).

A plurality of discrete macrocycles may self-assemble (e.g., stack together) through hydrogen bonding interactions into a three-dimensional crystalline structure. This structure may consist of alternating layers of macrocycles that stack in an a-b-c fashion. Each macrocycle forms a total of twelve hydrogen bonds (two per adeninate) with its six nearest-neighbor macrocycles within the structure. This packing motif (e.g., crystalline stacking) results in the formation of channels, such as pores and/or cylindrical cavities, arranged periodically throughout the three-dimensional structure. The confines of each cavity/pore are defined by one central macrocycle and fragments of the six nearest-neighbor macrocycles. In certain embodiments, the three-dimensional crystalline structure includes one-dimensional cavities/pores running along the c-crystallographic direction. The size, shape and number of the cavities/pores can vary. In one embodiment, the pores are approximately 6 angstroms in diameter. The unit cell parameters for the crystal structure are: R-3 a=b=17.74072 c=33.450; alpha=beta=90 gamma=120; cell volume=9117.3.) The molecular formula for the materials shown in FIG. 1 is as follows:

Zn₆(ad)₆(py)₆(ac)₆ and Zn₆(ad)₆(py)₆(cb)₆ (I)

wherein “Zn” represents zinc, “ad” represents adeninate, “ac” represents acetate, “py” represents pyridine, and “cb” represents carbamate.

In another embodiment, the invention includes materials having the following molecular formula:

Zn₈(ad)₄(BPDC)₆O.2NH₂(CH₃)₂,11H₂O,8DMF (II)

wherein “Zn” represents zinc, “ad” represents adeninate, “BPDC” represents 4,4-biphenyldicarboxylate, and “DMF” represents dimethylformamide.

The material having the molecular formula of (II) above is constructed from one-dimensional columns consisting of chains of corner-sharing zinc-adeninate octahedra. The columns are linked together into a crystalline three-dimensional porous framework using 4,4′-biphenyldicarboxylate linkers. This framework consists of metal-organic secondary building units (the zinc-adeninate octahedra). These units are then connected together with a biphenyldicarboxylate linker. This three-dimensional bio-structure contains large one-dimensional channels oriented along the c-crystallographic direction. Thus, the method of the invention can produce porous metal-organic frameworks having hierarchical porosity; e.g., pores within zinc adeninate octahedra and pores generated by linking the octahedra together with the biphenyldicarboxylate units. The unit cell parameters for the crystal structure are: I4(1)22 a=b=38.2372 c=11.1753; alpha=beta=gamma=90.

In accordance with certain embodiments of the invention, the material having the molecular formula of (II) may be subjected to cation exchange. The dimethylammonium cations located within the pores are exchanged with one or more of (i) tetramethylammonium cations, (ii) tetraethylammonium cations and (iii) tetrabutylammonium cations. Prior to the cation exchange, the pores containing dimethylammonium cations, may have a pore volume of about 0.75 cc/g and BET of about 1680 m²/g. When the dimethylammonium cations are exchanged with tetramethylammonium cations, the pore volume and BET can decrease to about 0.65 cc/g and 1460 m²/g, respectively. When the dimethylammonium cations are exchanged with tetraethylammonium cations, the pore volume and BET can decrease to about 0.55 cc/g and 1220 m²/g, respectively. When the dimethylammonium cations are exchanged with tetrabutylammonium cations, the pore volume and BET can decrease to about 0.37 cc/g and 830 m²/g, respectively.

FIG. 2 shows a framework in accordance with an embodiment of the invention wherein zinc-adeninate columnar building units are connected by biphenyldicarboxylate ligands within a crystal structure. The spheres indicate void (e.g., pore) space within the zinc-adeninate octahedra which form the zinc-adeninate columns.

In other embodiments, the invention includes materials having the following molecular formula:

Co₂(ad)₂(CO₂CH₃)₂.2DMF,0.5H₂O (III)

wherein “Co” represents cobalt, “ad” represents adeninate, and “DMF” represents dimethylformamide.

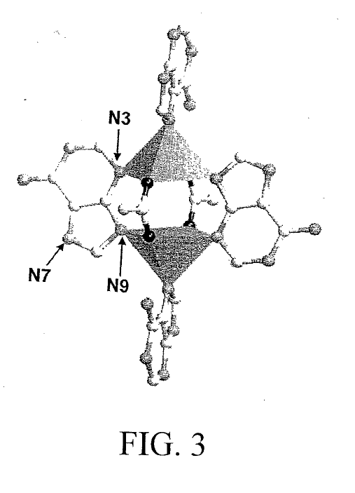

In certain embodiments, the material having molecular formula (III) is prepared via a solvothermal reaction between cobalt acetate tetrahydrate and adenine in N,N-dimethylformamide. FIG. 3 is shows the three-dimensional framework of a cobalt-adeninate metal-organic framework which exhibits pores that are densely lined with pyrimidal nitrogens and amino groups from the adenine building blocks, in accordance with certain embodiments of the invention. This material is constructed from cobalt-adeninate-acetate “paddle-wheel” clusters in which two Co²⁺ are each bridged by two adeninates via the N3 and N9 positions and two acetates in the dimonodentate alignment. These units are linked together through apical coordination of the adeninate N7 to Co²⁺ on neighboring clusters to generate a three-dimensional framework structure. There are open cavities/pores periodically distributed throughout the structure. The pores are densely lined with Lewis basic amino and pyrimidine groups, because the connectivity of the framework leaves these groups uncoordinated and exposed to the channels. The unit cell parameters for the crystal structure are: I41/A a=b=15.4355 c=22.7753; alpha=beta=gamma=90.

In other embodiments, the invention includes materials having the following molecular formula:

Zn₈(ad)₄(O)₂(BPDC)₆.4Me₂NH₂,xDMF,xH₂O (IV)

wherein “Zn” represents zinc, “ad” represents adeninate, “BPDC” represents 4,4-biphenyldicarboxylate, “Me” represents methyl, “DMF” represents dimethyl formamide, and “x” represents the number of molecules in the pore.

The material having the molecular formula of (IV) above is constructed from discrete zinc-adeninate octahedral cages. Three biphenyldicarboxylates coordinate to the Zn²⁺ tetrahedra on each open face of the octahedral cage and connect the octahedra together into a diamond-like network, where each zinc-adeninate octahedral cage serves as a tetrahedral four-connected building unit. Due to the length of the dicarboxylate linker and the size of the zinc-adeninate building unit, the resulting framework exhibits large mesoporous pores or cavities. This framework is also anionic and contains dimethylammonium cations within its pores. The unit cell parameters for the crystal structure are: IA-3D a=b=c=69.1153; alpha=beta=gamma=90.

The solid-state crystalline structure of the zinc-adeninate macrocycles contains cylindrical openings, such as pores, channels, or cavities which are about 22 angstroms in length and about 6 angstroms in diameter. The aperture to these pores is small, however, measuring only about 1-2 angstroms. The pyridine ligands constrict the entrance to these cavities. Without intending to be bound by any theory, it is believed that heating the materials may remove some of the pyridine ligands, thereby introducing an access route to the cylindrical cavities and therefore enabling the cavities to store and trap materials, as previously described. For example, it is believed that the cavities can be used to store hydrogen at low pressures because when hydrogen is introduced into the pores of the framework, it remains there until significant external vacuum is applied to release it from the pores. Storing hydrogen at low pressures is desirable for various applications, including, but not limited to, on-board hydrogen fueling applications and economic storage of large amounts of hydrogen in laboratory cylinders. Furthermore, the biomolecule-containing porous metal-organic frameworks of the invention could potentially be used for trapping CO₂. This is an important industrial application, in particular, for the removal of CO₂ from the emissions of various manufacturing or process facilities, such as, but not limited to, coal-fired power plants.

In one aspect of the invention, the biomolecule-containing porous metal-organic frameworks of the invention include anionic frameworks, and therefore, as previously mentioned, the cations located in the pores can be exchanged with other cations. For example, the zinc-adenine frameworks described above herein are anionic frameworks and therefore, the dimethylammonium cations present in the pores can be exchanged out. In general, cations are mobile and can be removed and replaced with a variety of other cationic molecules via simple cation exchange. In one embodiment, the cation can be used to balance the charge. Without intending to be bound by any particular theory, it is believed that the pore size or volume and pore functionality of the framework can be modified or adjusted by introducing different cations into the pores. For example, replacing at least a portion of the cations in the pores with larger or smaller cations can result in a modified, increased or decreased, capacity of the pores. Thus, the pores can adsorb a greater or lesser amount of materials. In one embodiment, the dimethylammonium cations in the pores can be exchanged with tetramethylammonium cations (e.g., slightly larger cations). The introduction of these slightly larger cations can modify the pore space and the amount of material that may be contained or stored in the pore. In an embodiment, a larger pore space can accommodate up to 100 cm³/g of carbon dioxide. Further, it is believed that constriction of the pore space may allow for increased inter-sorbent interactions and therefore, a higher capacity for storage.

The cobalt-adenine frameworks described above exhibit Lewis basic groups exposed to the pore channels. These groups are optimal for interacting with CO₂. The CO₂ isotherm is also depicted as a reversible type I isotherm and adsorption and desorption cycles can be repeated with complete reversibility. For example, at 1 bar, this material is capable of adsorbing 6.0 mmol/g at 273K and 4.1 mmol/g at 298K. However, its N₂ uptake at 273K and 298K has been found to be only 0.43 mmol/g and 0.13 mmol/g, respectively, indicating that the pores preferentially adsorb CO₂ over N₂. The isoteric heat of CO₂ sorption is calculated from the Clausius-Clapeyron equation.

In another aspect of the invention, the biomolecule-containing porous metal-organic frameworks include neutral frameworks, and therefore, as previously mentioned, a material having the group OOC—R can be selected such that pore size, pore volume and functionality of the MOF may be controlled. For example, in accordance with certain embodiments of the invention, a neutral framework of the following molecular formula is formed:

M₂(ad)₂(OOC—R)₂ (V)

wherein “M” represents a metal, such as Cu, Ti, Cr, Fe, Ni, Mn, Co, Zn, Zr, Al, In, and salts and mixtures thereof, “ad” represents adeninate which is the deprotonated form of the biomolecule adenine, and “OOC—R” represents the deprotonated form of a monocarboxylic acid, wherein “R” represents an organic group such as branched or un-branched alkyl and in certain embodiments, C₁-C₆ alkyl. As previously mentioned, non-limiting examples of suitable monocarboxylates include acetate, propionate, butyrate, valerate, isobutyrate, isovalerate, R-2-methylbutyrate, S-2-methylbutyrate, pivalate, 2-cyclopropylacetate, 2-methylcyclopropane-1-carboxylate, R/S-2-methylbutyrate, cyclobutanecarboxylate, and mixtures thereof. In certain embodiments, R may include a functional group, such as but not limited to, hydroxy (OH). The oxygen substituent is pendent to the first carbon (C₁) in the carbon chain.

Further, without intending to be bound by any particular theory, it is believed that R is a pendant group which dangles into the pores of a crystal structure, defining the pore space or volume and functionality. The pore space or volume can be tuned, e.g., increased or decreased, and gas adsorption, e.g., carbon dioxide (CO₂) capture, properties may be adjusted. For example, by selecting a specific aliphatic carbon chain, pore spaces may be created which have a high affinity for CO₂ at low pressure, such as less than about 0.1 bar, and high temperature, such as greater than about 298K. In another example, by selecting a specific aliphatic carbon chain, the stability of the MOF in moist environments may be tuned, such that for instance, the MOF may withstand soaking in water for an extended period of time, e.g., for weeks, without experiencing loss of adsorption capacity. These properties are particularly desirous for CO₂ capture from flue gas of coal fired power plants (because flue gas has significant water content).

In certain embodiments, the R substituent may be selected such that isosteric heat of adsorption for CO₂ is optimized, e.g., about 45 kJ/mol. This property allows the MOF interactions to be sufficiently large to capture CO₂ efficiently and not too large that the CO₂ cannot be desorbed. There are known adsorbents which have high initial heats of adsorption, e.g., at zero coverage of CO₂, but which rapidly decline as CO₂ loading increases. In accordance with the invention, MOFs have been found that demonstrate constant or nearly constant isosteric heat from low loading to high loading.

In accordance with the invention, the pore size, shape, and functionality of the porous metal-organic frameworks may be modified, adjusted and controlled. Therefore, the frameworks can be used as a scaffold to produce a wide variety of functionally unique materials by selecting various species to be introduced into the framework. This capability is useful for a wide variety of applications, such as, but not limited to, gas storage, drug delivery (e.g., controlled release), carbon dioxide capture, and the like. Since the porous metal-organic frameworks include those constructed in part from biomolecular components, the frameworks can be particularly suitable for drug storage/release applications as compared to these frameworks in the prior art that may not be biologically compatible.

The materials of the invention are stable in a variety of organic solvents, water, and biological buffers, such that they can be used in various types of environments. For example, the frameworks can remain crystalline in solvents, such as, but not limited to, water, acetonitrile, chloroform, ethanol and mixtures thereof. In another example, the frameworks can remain crystalline in biological buffers for several weeks or longer. Further, the materials of the invention demonstrate thermal stability at a variety of temperature conditions. For example, in certain embodiments, the frameworks can remain crystalline up to a temperature of 250° C. The frameworks are also essentially permanently porous with a BET surface area ranging from about 1000 to about 4500 m²/g or from about 2000 to about 4500 m²/g, based on gas sorption measurements.

In non-limiting embodiments, the chemistry of the pores can be modified for specific applications, such as CO₂ capture or separations or gas and liquid separations.

In certain embodiments, the invention provides the ability to selectively assemble biological components into hierarchical structures using metal ions. Biomolecules can be used as building blocks for porous metal-organic crystalline materials. Biomolecules can be bio-compatible and recyclable. These characteristics are desirous for environmental applications (e.g., environmental cleanup/remediation), food industry applications (e.g., food fresheners/deodorizers), and biomedical applications (e.g., nitric oxide delivery, drug delivery, and enzyme sequestration). For example, in drug delivery applications, the porous metal-organic frameworks of the invention remain stable throughout the duration of drug release and then they biodegrade after complete release of drug.

In certain embodiments, wherein the porous biomolecule-containing metal-organic framework has been subjected to a cation exchange process and/or formed by the selection of a monocarboxylate material, and the pores are at least partially loaded with material (e.g., different material than the material that was originally in the pores when the framework was formed), the framework can be soaked in a buffer solution, such as, but not limited to, phosphate buffered saline. In this embodiment, the material which is at least partially loaded in the pores can include drug molecules for a drug delivery application.

Porous materials generally can be used as a mechanism for release agents, such as but not limited to drug molecules, because their pore size and functionality can be controlled and adjusted based on the release profile of particular drug molecules. Porous materials constructed from molecular building blocks and metal ions are particularly useful because 1) the pore metrics and functionality can be systematically tuned and 2) they can be constructed from biomolecules and biologically-compatible metal ions. Therefore, materials can be designed and created to be biodegradable and how they interact with and store drug molecules can be substantially controlled.

The porous biomolecule-containing metal-organic frameworks of the invention demonstrate at least one of the following advantages: 1) high permanent porosity; 2) high stability under a wide variety of conditions, including biological and environmental media; 3) ability to vary pore functionality by performing facile cation-exchange experiments; 4) high affinity for CO₂ and 5) ability to maintain crystalline integrity throughout the release of material from pores therein, however, following complete release of material, ability to lose crystalline integrity and biodegrade.

### EXAMPLES

### Example 1

### Zn₆(ad)₆(py)₆(cb)₆, H₂ and CO₂ Sorption Studies

A mixture of adenine and zinc nitrate was dissolved in dimethylformamide (DMF) and pyridine. Heating this solution resulted in the formation of a solid-state crystalline structure of zinc-adeninate macrocycles having cylindrical cavities which were approximately 22 angstroms in length and 6 angstroms in diameter. The aperture to these cavities was small, measuring only approximately 1-2 angstroms. The Zn6(adeninate)6(py)6(cb)6 was heated (e.g., activated) to 125° C., some of the pyridine molecules were removed and gases then accessed the pores. This was evidenced by gas sorption studies performed on the material. The activated material adsorbed 2.19 weight percent H₂ at 77 K and 760 mmHg, which compared favorably to other similar porous molecular materials. Significant hysteresis was observed when we monitored H₂ desorption. Approximately 1% by weight H₂ remained trapped within the material at pressures as low as 50 torr. Thus, it is believed that this type of material may be suitable to store hydrogen at low pressures because once the hydrogen was introduced into the pores of the material, it remained there until significant external vacuum was applied to remove it.

The CO₂ sorption properties of this material were also tested. The CO₂ sorption isotherm showed similar hysteresis as demonstrated for H₂, although not as dramatic. Therefore, this material and others like it may be suitable for trapping CO₂.

### Example 2

### Zn₈(ad)₄BPDC₆O″ 2NH₂(CH₃)₂, 11 H₂O, 8 DMF: Cation Exchange and CO₂ Sorption Studies

A mixture of adenine and zinc acetate dihydrate in dimethylformamide (DMF) and biphenyl dicarboxylic acid was prepared, and an anionic structure was formed having molecular structure Zn₈(ad)₄(BPDC)₆O″ 2NH₂(CH₃)₂, 11 H₂O, 8 DMF. Elemental analysis (EA) and thermogravimetric analysis (TGA) demonstrated that dimethylammonium cations (the product of DMF decomposition) as well as DMF and water resided in the pores of the structure. The dimethylammonium cations were exchanged out of the material via cation exchange. The potential to modify the pore size and functionality by introducing different cations into the pores was evaluated. In particular, it was shown that by introducing larger organic cations into the pores, the CO₂ capacity of the pores was increased. The CO₂ uptake of the evacuated as-synthesized material which contains dimethylammonium cations within the pores was measured. It was found that this material adsorbed 77 cm³/g of CO₂ at 0° C. This capacity for CO₂ was favorable. To determine whether the capacity of this material for CO₂ could be controlled, the dimethylammonium cations in the pores were exchanged with tetramethylammonium cations. Introducing this slightly larger cation modified the pore space and allowed it to accommodate a larger amount of CO₂, up to 100 cm³/g. Without intending to be bound by any theory, it was believed that the constriction of the pore space may allowed for greater inter-sorbent interactions and thus a higher capacity for storage.

### Example 3

### Zn₈(ad)₄(BPDC)₆O″ 2NH₂(CH₃)₂, 11 H₂O, 8 DMF: Drug Storage/Release Studies

Zn₈(ad)₄(BPDC)₆O″ 2NH₂(CH₃)₂, 11 H₂O, 8 DMF, as prepared in accordance with the description in Example 2, was evaluated for its capacity to store and release drug molecules. Zn₈(ad)₄(BPDC)₆O″ 2NH₂(CH₃)₂, 11 H₂O, 8 DMF, being an anionic material, was loaded with cationic drug molecules by soaking the material in water to remove all the DMF molecules and then performing cation exchange experiments to replace the dimethylammonium cations with cationic drug molecules. The drug release was then controlled through cation exchange with metal ions such as Mg²⁺ or Na⁺, which are common in biological systems. The capture and release of procainamide, a cationic drug molecule which is a class IA antiarhythmic drug used for life-threatening or symptomatic ventricular arrhythmias, was evaluated. One problem with procainamide is that it rapidly metabolizes and it has an elimination half-life of approximately 3 hours. It was a good candidate for controlled release, as it allowed for protracted dosing and protection of the procainamide from degradation during the dosing period.

The water-exchanged material was soaked with aqueous solutions of procainamide-hydrochloric acid (HCl) to load the pores of the material with procainamide molecules. Complete loading was confirmed by TGA, EA, and gas sorption experiments. To evaluate the release profile of the adsorbed procainamide and whether its release could be controlled via cation exchange with metal cations present in biological buffers, the procainamide-exchanged material was soaked in 0.1 M phosphate-buffered saline (PBS) having a pH of 7.4. The release of procainamide was monitored via high-pressure liquid chromatography (HPLC). Steady procainamide release was observed over approximately a 20-hour period, and total release of the procainamide was completed after approximately 72 hours. The crystalline integrity of the framework was maintained throughout the release process, as evidenced by X-ray powder diffraction experiments. However, after all of the drug molecules had been released, the material began to lose its crystalline integrity and appeared to break down in the biological buffers. This feature may be useful in for drug-delivery/controlled release applications since it would be desirable for the material to maintain its integrity during the release time of the drug and then after release for the material to degrade and be flushed from the body.

### Example 4

### Preparation of Cobalt Salts

Freshly prepared cobalt carbonate was made from mixing cobalt nitrate aqueous solution (1.5 mL; 1 M) and sodium carbonate aqueous solution (1.5 mL; 1 M). The precipitates were washed with water (×3) and ethanol (×2), and subsequently aliphatic acid (1.5 mmol, 1 equivalent) was added. Additional water (minimal amount) and ultrasonication were used to fully dissolve the cobalt carbonate. Once a clear cobalt carboxylate solution was formed, the vial was placed in a 100° C. oven for several hours to yield dry cobalt carboxylate solids.

### Single Crystal X-Ray Diffraction

The crystal structures of the analogues were determined by single crystal X-ray diffraction experiments. Single crystal X-ray diffraction data was collected on a Bruker X8 Prospector Ultra equipped with an Apex II CCD detector and an IμS micro-focus CuK\α X-raysource (λ=1.54178 Å). A single crystal of each MOF was mounted onto a MiTeGen micromount with fluorolube. The data was collected under ambient temperature.

### Gas Adsorption Studies

Gas adsorption isotherms were collected using a Quantachrome Autosorb-1 instrument. As-synthesized crystals were thoroughly washed with anhydrous dichloromethane and dried under argon flow. Approximately 60 mg of each sample was added into a pre-weighed sample analysis tube. The samples were degassed at 100° C. under vacuum for 24-48 hours until the pressure change rate was no greater than 3.5 mTorr/min. A liquid N₂ bath was used for the N₂ adsorption experiments at 77 K. A water/ethylene glycol bath was used for isotherms collected at 273 K, 298 K, 303 K, 308 K, and 313 K. BET surface areas were calculated using N₂ isotherms at 77 K. UHP grade N₂ and CO₂ gas adsorbates (99.999%) were used.

### Syntheses of Analogues and Corresponding Monocarboxylates Employed

### Acetate Analogue

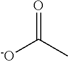

Stock solutions of cobalt acetate (0.05 M) and adenine (0.05 M) in pre-dried DMF were prepared. To a Schlenk tube (40 mL) were added cobalt acetate solution (9.0 mL; 0.45 mmol), adenine solution (27.0 mL; 1.35 mmol), and nanopure water (120 μL). After the solution was frozen in liquid nitrogen and evacuated to 200 mTorr, it was heated in a 130° C. oven (24 h). Black, octahedral crystals were collected, washed (dry DMF, 3×), and dried under argon flow. Yield: 102 mg, 90% (based on cobalt acetate salt). Anal. Calcd. for Co₂(ad)₂(CH₃CO₂)₂.2.25 DMF, 0.6H₂O (ad=adeninate): C, 36.68; H, 4.59; N, 25.25. Found: C, 36.70; H, 4.06; N, 25.205.

Crystal system: Tetragonal.

Space group: I4(1)/a.

Unit cell dimensions: a=b=15.4355 (18), c=22.775 (5), α=β=γ=90°

Final R indices [I>2sigma(I)]: R1=0.1047, wR2=0.2834

R indices (all data): R1=0.1371, wR2=0.2974

### Propionate Analogue

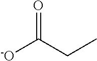

Stock solutions of cobalt propionate (0.05 M) and adenine (0.05 M) in pre-dried DMF were prepared. To a Schlenk tube (40 mL) were added cobalt propionate solution (9.0 mL; 0.45 mmol), adenine solution (27.0 mL; 1.35 mmol), and nanopure water (120 μL). After the solution was frozen in liquid nitrogen and evacuated to 200 mTorr, it was heated in a 130° C. oven (24 h). Black, octahedral crystals were collected, washed (dry DMF, 3×), and dried under argon flow. Yield: 94 mg, 78% (based on Co propionate). Anal. Calcd. for Co₂(ad)₂(C₂H₅CO₂)₂.2.25 DMF, 0.3H₂O: C, 38.92; H, 4.93; N, 24.44.

Found: C, 38.99; H, 4.59; N, 24.36.

Crystal system: Tetragonal.

Space group: I4(1)/a.

Unit cell dimensions: a=b=17.243 (3), c=20.157 (6), α=β=γ=90°

Final R indices [I>2sigma(I)]: R1=0.0474, wR2=0.0855

R indices (all data): R1=0.0899, wR2=0.0985

### Butyrate Analogue

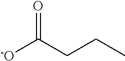

Stock solutions of cobalt butyrate (0.05 M) and adenine (0.05 M) in pre-dried DMF were prepared. To a Schlenk tube (40 mL) were added cobalt valerate solution (9.0 mL; 0.45 mmol), adenine solution (27.0 mL; 1.35 mmol), and nanopure water (120 μL). After the solution was frozen in liquid nitrogen and evacuated to 200 mTorr, it was heated in a 130° C. oven (24 h). Black, octahedral crystals were collected, washed (dry DMF, 3×), and dried under argon flow. Yield: 90 mg, 71% (based on Co butyrate). Anal. Calcd for Co₂(ad)₂(C₃H₇CO₂)₂1.1 DMF, 0.6H₂O: C, 39.27; H, 4.78; N, 23.86. Found: C, 39.36; H, 4.185; N, 23.74.

Crystal system: Tetragonal.

Space group: I4(1)/a.

Unit cell dimensions: a=b=15.7869 (10), c=22.328 (3), α=β=γ=90°

Final R indices [I>2sigma(I)]: R1=0.0541, wR2=0.1677

R indices (all data): R1=0.0687, wR2=0.1836

### Valerate Analogue

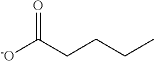

Stock solutions of cobalt valerate (0.05 M) and adenine (0.05 M) in pre-dried DMF were prepared. To a Schlenk tube (40 mL) were added cobalt valerate solution (9.0 mL; 0.45 mmol), adenine solution (27.0 mL; 1.35 mmol), and nanopure water (120 μL). After the solution was frozen in liquid nitrogen and evacuated to 200 mTorr, it was heated in a 130° C. oven (24 h). Black, octahedral crystals were collected, washed (dry DMF, 3×), and dried under argon flow. Yield: 85 mg, 64% (based on Co valerate). Anal. Calcd for Co₂(ad)₂(C₄H₉CO₂)₂.0.6 DMF, 0.6H₂O: C, 40.72; H, 4.92; N, 23.09. Found: C, 40.8; H, 4.63; N, 22.93.

Crystal system: Tetragonal.

Space group: I4(1)/a.

Unit cell dimensions: a=b=15.852 (3), c=22.346 (8), α=β=γ=90°

Final R indices [I>2sigma(I)]: R1=0.0658, wR2=0.1834

R indices (all data): R1=0.1442, wR2=0.2298

### Isobutyrate Analogue

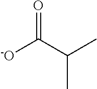

Stock solutions of cobalt isobutyrate (0.05 M) and adenine (0.05 M) in pre-dried DMF were prepared. To a Schlenk flask (50 mL) were added cobalt isobutyrate solution (9 mL; 0.45 mmol), adenine solution (18 mL; 0.9 mmol), and nanopure water (90 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

Crystal system: Tetragonal.

Space group: I4(1)/a.

Unit cell dimensions: a=b=17.0103 (8), c=21.0175 (10), α=β=γ=90°

Final R indices [I>2sigma(I)]: R1=0.0580, wR2=0.1732

R indices (all data): R1=0.0732, wR2=0.1874

### 2-Cyclopropanecarboxylate Analogue

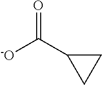

Stock solutions of cobalt cyclopropanecarboxyate (0.05 M) and adenine (0.05 M) in pre-dried DMF were prepared. To a Schlenk flask (50 mL) were added cobalt cyclopropanecarboxylate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

Crystal system: Tetragonal.

Space group: I4(1)/a.

Unit cell dimensions: a=b=17.1517 (8), c=20.3633 (13), α=β=γ=90°

Final R indices [I>2sigma(I)]: R1=0.0557, wR2=0.1704

R indices (all data): R1=0.0671, wR2=0.1792

### Isovalerate Analogue

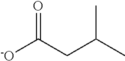

Stock solutions of cobalt isovalerate (0.05 M) and adenine (0.05 M) in pre-dried DMF were prepared. To a Schlenk flask (50 mL) were added cobalt isovalerate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

Crystal system: Tetragonal.

Space group: I4(1)/a.

Unit cell dimensions: a=b=15.6075 (6), c=22.5291 (13), α=β=γ=90°

Final R indices [I>2sigma(I)]: R1=0.0617, wR2=0.1587

R indices (all data): R1=0.1234, wR2=0.1873

### R-2-Methylbutyrate Analogue

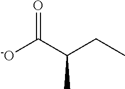

Stock solutions of cobalt R-2-methylbutyrate (0.05 M) and adenine (0.05 M) in pre-dried DMF were prepared. To a Schlenk flask (50 mL) were added cobalt R-2-methylbutyrate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

### S-2-Methylbutyrate Analogue

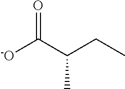

Stock solutions of cobalt S-2-methylbutyrate (0.05 M) and adenine (0.05 M) in pre-dried DMF were prepared. To a Schlenk flask (50 mL) were added cobalt S-2-methylbutyrate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

Crystal system: Tetragonal.

Space group: I4(1)/a.

Unit cell dimensions: a=b=16.4611 (4), c=20.7420 (12), α=β=γ=90°

Final R indices [I>2sigma(I)]: R1=0.0499, wR2=0.1201

R indices (all data): R1=0.0742, wR2=0.1316

### R/S-2-Methylbutyrate Analogue

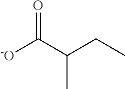

Stock solutions of cobalt R/S-2-methylbutyrate (0.05 M) and adenine (0.05 M) in pre-dried DMF were prepared. To a Schlenk flask (50 mL) were added cobalt R/S-2-methylbutyrate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24-48 hours. Black octahedral crystals were collected and washed with dry DMF.

Crystal system: Tetragonal.

Space group: I4(1)/a.

Unit cell dimensions: a=b=16.4437 (8), c=20.8020 (3), α=β=γ=90°

Final R indices [I>2sigma(I)]: R1=0.0506, wR2=0.1187

R indices (all data): R1=0.0839, wR2=0.1376

### Pivalate Analogue

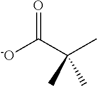

Stock solutions of cobalt pivalate (0.05 M) and adenine (0.05 M) in pre-dried DMF were prepared. To a Schlenk flask (50 mL) were added cobalt pivalate solution (9 mL; 0.45 mmol), adenine solution (18 mL; 0.9 mmol), and nanopure water (180 μL). After the mixture was frozen in liquid nitrogen, evacuated to 200 mTorr and warmed up to room temperature, it was heated in a 130° C. oven for 48-72 hours. Black octahedral crystals were collected and washed with dry DMF.

Crystal system: Tetragonal.

Space group: I4(1)/a.

Unit cell dimensions: a=b=16.9349 (7), c=20.3498 (8), α=β=γ=90°

Final R indices [I>2sigma(I)]: R1=0.0695, wR2=0.1730

R indices (all data): R1=0.0967, wR2=0.1946

### 2-Cyclopropylacetate Analogue

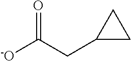

Stock solutions of cobalt 2-cyclopropylacetate (0.05 M) and adenine (0.05 M) in pre-dried DMF were prepared. To a Schlenk flask (50 mL) were added cobalt 2-cyclopropylacetate solution (9 mL; 0.45 mmol), adenine solution (18 mL; 0.9 mmol), and nanopure water (60 μL). After the mixture was frozen in liquid nitrogen, evacuated to 200 mTorr and warmed up to room temperature, it was heated in a 130° C. oven for 24 hours. Black octahedral crystals were collected and washed with dry DMF.

Crystal system: Tetragonal.

Space group: I4(1)/a.

Unit cell dimensions: a=b=15.6392 (8), c=22.5622 (13), α=β=γ=90°

Final R indices [I>2sigma(I)]: R1=0.0589, wR2=0.1708

R indices (all data): R1=0.0801, wR2=0.1882

### 2-Methylcyclopropane-1-Carboxylate Analogue

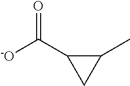

Stock solutions of cobalt 2-methylcyclopropane-1-carboxylate (0.05 M) and adenine (0.05 M) in pre-dried DMF were prepared. To a Schlenk flask (50 mL) were added cobalt 2-methylcyclopropane-1-carboxylate solution (9 mL; 0.45 mmol), adenine solution (18 mL; 0.9 mmol), and nanopure water (180 μL). After the mixture was frozen in liquid nitrogen, evacuated to 200 mTorr and warmed up to room temperature, it was heated in a 130° C. oven for 48-72 hours. Black octahedral crystals were collected and washed with dry DMF.

Crystal system: Tetragonal.

Space group: I4(1)/a.

Unit cell dimensions: a=b=16.7580 (2), c=21.0735 (4), α=β=γ=90°

Final R indices [I>2sigma(I)]: R1=0.0526, wR2=0.1696

R indices (all data): R1=0.0682, wR2=0.1827

### Cyclobutanecarboxylate Analogue

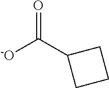

Stock solutions of cobalt cyclobutanecarboxylate (0.05 M) and adenine (0.05 M) in pre-dried DMF were prepared. To a Schlenk flask (50 mL) were added cobalt cyclobutanecarboxylate solution (9 mL; 0.45 mmol), adenine solution (18 mL; 0.9 mmol), and nanopure water (120 μL). After the mixture was frozen in liquid nitrogen, evacuated to 200 mTorr and warmed up to room temperature, it was heated in a 130° C. oven for 24 hours. Black octahedral crystals were collected and washed with dry DMF.

Crystal system: Tetragonal.

Space group: I4(1)/a.

Unit cell dimensions: a=b=17.5889 (14), c=20.5290 (5), α=β=γ=90°

Final R indices [I>2sigma(I)]: R1=0.0724, wR2=0.1797

R indices (all data): R1=0.1275, wR2=0.2098

### Cyclobutanecarboxylate Analogue

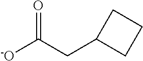

Stock solutions of cobalt cyclobutylacetate (0.05 M) and adenine (0.05 M) in pre-dried DMF were prepared. To a glass tube were added cobalt cyclobutylacetate solution (0.3 mL; 0.015 mmol), adenine solution (0.6 mL; 0.03 mmol), and nanopure water (14 μL). After the mixture was frozen in liquid nitrogen, evacuated to 200 mTorr and warmed up to room temperature, it was heated in a 130° C. oven for 72-96 hours. Black octahedral crystals were collected and washed with dry DMF.

Crystal system: Tetragonal.

Space group: I4(1)/a.

Unit cell dimensions: a=b=15.9282 (20), c=22.3953 (31), α=β=γ=90°

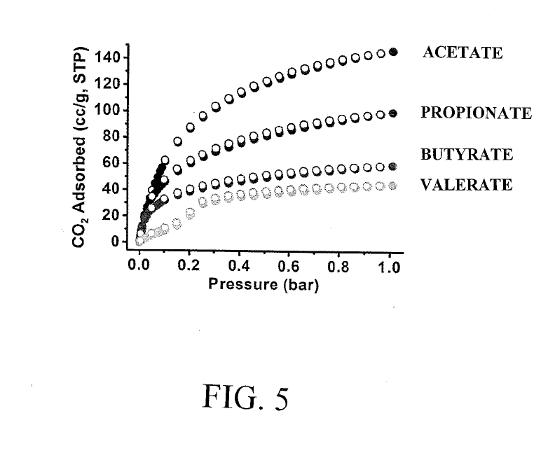

FIG. 5 shows carbon dioxide adsorption isotherms for the above described acetate, propionate, butyrate and valerate analogues at 273 K. As shown in FIG. 5, at 273 K and 1 bar, the acetate, propionate, butyrate and valerate analogues each adsorb 147 cc/g carbon dioxide, 100 cc/g carbon dioxide, 60 cc/g carbon dioxide, and 45 cc/g carbon dioxide, respectively.

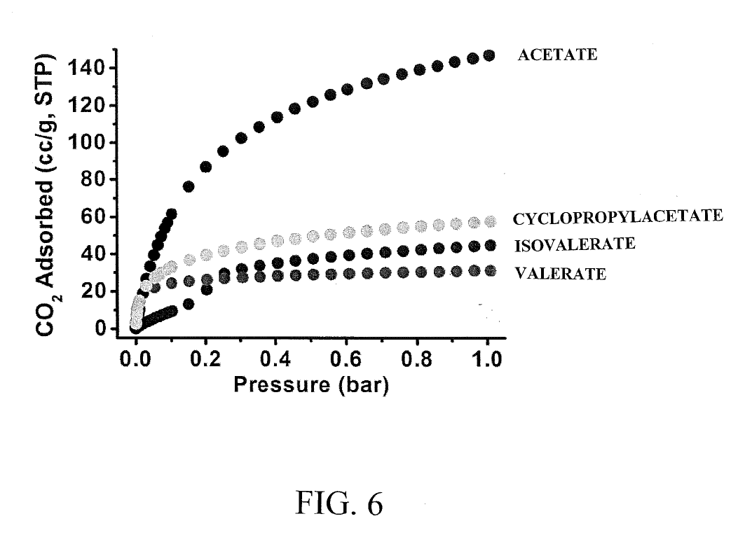

FIG. 6 shows carbon dioxide adsorption isotherms for the above described acetate, cyclopropylacetate, isovalerate and valerate analogues at 273 K. The data in FIG. 6 shows that the selection of the aliphatic carboxylate affects the gas adsorption properties. The capacity for carbon dioxide varies as a function of aliphatic carboxylate.

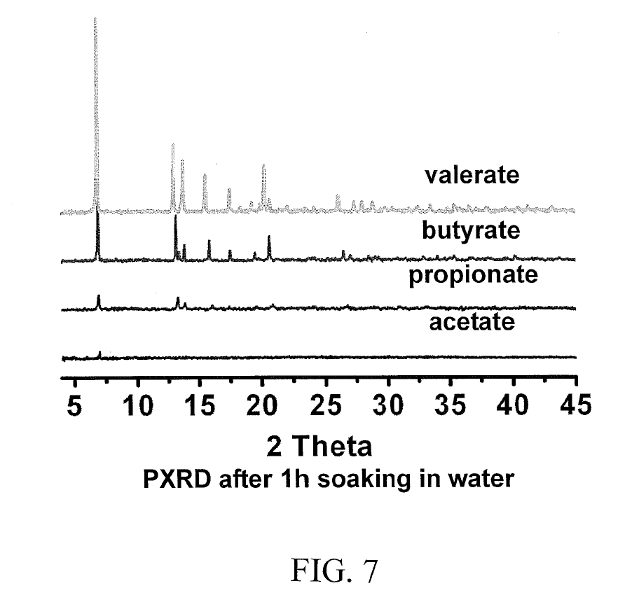

FIG. 7 shows powder x-ray diffraction data after soaking in water for one hour the above described acetate, propionate, butyrate and valerate analogues. FIG. 7 shows that the selection of the aliphatic monocarboxylate affects the material stability in water. As the aliphatic chain increases in length, the water stability increases. The valerate analogue maintains its crystallinity while the propionate and acetate analogues lose their crystallinity, as evidenced by a loss of diffraction lines in the XRD pattern.

### Example 5

### Synthesis of Bio-MOF-100

A stock solution of adenine (0.05 M) in N,N-dimethylformamide (DMF) was prepared. Ultra-sonication and heating was employed to completely dissolve adenine in DMF. A stock solution of zinc acetate dihydrate (0.05 M) and 4,4′-biphenyldicarboxylic acid (0.1 M) in DMF was prepared. Adenine stock solution (2.5 mL, 0.125 mmol), zinc acetate dihydrate stock solution (5 mL, 0.25 mmol) and 4,4′-biphenyldicarboxylic acid stock solution (2.5 mL, 0.25 mmol) were added to a 20 mL vial. 2.5 mL of DMF, 1 mL of methanol, and 0.25 mL of nanopure water were subsequently added. The vial was capped and heated at 85° C. for 24 hours and then cooled to room temperature. The product colorless cubic crystals were washed with DMF (3 mL×3) and dried under Ar gas (30 min) (yield: 0.218 g, 25.4% based on adenine). FT-IR: (KBr 4000-400 cm⁻¹): 3341.18 (br), 3185.33 (br), 2929.70 (w), 1669.40 (s), 1607.51 (s), 1547.96 (w), 1467.74 (w) 1386.25 (s), 1255.31 (w), 1212.29 (m), 1176.27 (w), 1152.77 (m), 1097.54 (m), 855.89 (m) 843.48 (m), 773.25 (s).

Elemental analysis C₂₅₉H₅₀₁N₇₃O₁₀₆Zn₈=Zn₈(ad)₄(BPDC)₆, O₂.4Me₂NH₂, 49DMF, 31H₂O Calcd. C, 45.37; H, 7.36; N, 14.91. Found C, 45.43; H, 7.51; N, 14.84.

### Gas Adsorption Experiments

### Activation Via Supercritical Drying.

The general supercritical drying method is performed according to published procedures. The samples were activated with supercritical CO₂ in a Tousimis™ Samdri® PVT-30 critical point dryer. Prior to drying, the DMF solvated samples were soaked in absolute ethanol (EtOH), replacing the soaking solution for 48 hrs, to exchange the occluded solvent for EtOH. After the EtOH exchange process was complete the samples were placed inside the dryer and the EtOH was exchanged with CO_{2(L)} over a period of 8 hrs. During this time, the liquid CO₂ was vented under positive pressure for five minutes each hour. The rate of venting of CO_{2(L)} was always kept below the rate of filling so as to maintain a full drying chamber. After 8 hrs of venting and soaking with CO_{2(L)} the chamber was sealed and the temperature was raised to 40° C. This brought the chamber pressure to around 90 bar, i.e. above the critical point of CO₂. The chamber was vented over the course of 12-18 hrs. The dried samples were placed in sealed containers and stored in a desiccator.

Samples of the supercritical CO₂ dried materials were weighed using an AB54-S/FACT (Mettler Toledo) electrogravimetric balance (sensitivity 0.1 mg). 9 mm large bulb cells (from Quantachrome) of a known weight were loaded with ˜50 mg of sample for gas sorption experiments. The samples were degassed at rt for 24 hours on degassing station until the outgas rate was no more than 3.5 mTorr/min. The degassed sample and sample cell were weighed precisely and then transferred back to the analyzer. The temperature of each sample for N₂ adsorption experiments was controlled by a refrigerated bath of liquid nitrogen (77 K) The N₂, adsorbate was of UHP grade.

The nitrogen gas adsorption experiments revealed that the surface area of the material was 4300 m²/g and the pore volume was determined to be 4.3 cc/g.

Whereas particular embodiments of the invention have been described herein for purposes of illustration, it will be evident to those skilled in the art that numerous variations of the details may be made without departing from the invention as set forth in the appended claims.

1. A biomolecule-containing metal-organic framework having pores structured therein, said framework comprising a metal component and a biomolecule component, said pores structured to have a pore volume and to adsorb material therein, and said pore volume adjusted by a mechanism selected from the group consisting of cation exchange of cations in the pores with different cations and pre-selection of a pendant group R on an aliphatic chain of a framework comprising monocarboxylate having a structure (M)₂(BC)₂(OOC—R)₂ wherein M is the metal component, BC is the biomolecule component and R is branched or un-branched alkyl.

2. The framework of claim 1, wherein the metal component is selected from the group consisting of metal cluster, metal ion or combination thereof.

3. The framework of claim 1, wherein the metal component is selected from the group consisting of Cu, Ti, Cr, Fe, Ni, Mn, Co, Zn, Zr, Al, In, and salts and mixtures thereof.

4. The framework of claim 1, wherein, R is C₁ to C₅ alkyl.

5. The framework of claim 1, wherein the biomolecule is a nucleobase.

6. The framework of claim 5, wherein the nucleobase is selected from the group consisting of adenine, guanine, cytosine, thymine, uracil and mixtures thereof.

7. The framework of claim 1, further comprising an organic ligand.

8. The framework of claim 7, wherein the organic ligand is selected from the group consisting of dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures and combinations thereof.

9. The framework of claim 1, wherein the framework is selected from the group of structures consisting of macrocyclic structures, crystalline structures, polyhedra, extended framework structures and combinations thereof.

10. The framework of claim 1, wherein the framework comprises a plurality of units connected by a linker and wherein the linker is selected from the group consisting of 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid; 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; azobenzene-4,4′-benzenedicarboxylate; 4′,4,4″-[benzene-1,3,5-triyl-tris(benzene-4,1-diyl)]tribenzoate; and mixtures thereof.

11. The framework of claim 1, wherein the metal component comprises zinc salt or a cobalt salt.

12. The framework of claim 5, wherein the nucleobase comprises adenine.

13. The framework of claim 1, wherein the material adsorbed is selected from the group consisting of gas, drug, protein, polymer, and combinations thereof.

14. The framework of claim 13, wherein the gas is selected from the group consisting of carbon dioxide, hydrogen, nitrogen and mixtures thereof.

15. The framework of claim 13, wherein the material is drug and the framework is capable of a controlled release of said drug from said pores.

16. The framework of claim 1, wherein the biomolecule-containing metal-organic framework has a pore volume from about 2 to about 6 cm³/g.

17. A method for adjusting volume of pores formed in a biomolecule-containing metal-organic framework, comprising:
preparing a biomolecule-containing metal-organic framework having pores structured therein, said framework comprising a metal component and a biomolecule component, said pores structured to have a pore volume and to adsorb material therein; and
adjusting said volume by a mechanism selected from the group consisting of cation exchange of cations in the pores with different cations and pre-selection of a pendant group R on an aliphatic chain of a framework comprising monocarboxylate having a structure (M)₂(BC)₂(OOC—R)₂ wherein M is the metal component, BC is the biomolecule component and R is branched or un-branched alkyl.

18. A method for storing and controllably releasing drug material comprising the composition of claim 1.

19. A method for capturing and storing carbon dioxide material comprising the composition of claim 1.
